(19) United States
(12) Patent Application Publication
(10) Pub. No.: US 2015/0148603 A1
(43) Pub. Date: May 28, 2015

(54) Imaging Catheter with Thermal Management Assembly

(71) Applicant: Covidien LP, Mansfield, MA (US)

(72) Inventor: John Holste, Hamburg, IL (US)

(21) Appl. No.: 14/553,231

(22) Filed: Nov. 25, 2014

Related U.S. Application Data
(60) Provisional application No. 61/908,284, filed on Nov. 25, 2013.

(51) Int. Cl. A61B 1/12; A61B 1/015; A61J 15/00; A61B 1/005; A61B 1/06; A61B 1/00; A61B 1/273; A61B 1/05; G01K 13/00

(52) U.S. Cl. CPC A61B 1/128; A61B 1/051; A61B 1/015; A61J 15/0003; A61J 15/0026; G01K 13/002; A61B 1/0676; A61B 1/00006; A61B 1/00128; A61B 1/273; A61B 1/005; A61J 2015/0084; A61J 2015/0088
USPC 600/109

(57) ABSTRACT

An imaging catheter assembly that includes an elongate body having a first body end, and an opposite second body end; an imaging assembly secured to the first body end, the imaging assembly having a first imaging assembly end remote from the first body end and a second imaging assembly end adjacent the first body end, the imaging assembly including a flex circuit having an electronic component mounting portion, a camera mounting portion adjacent the first imaging assembly end, and a light mounting portion adjacent the first imaging assembly end; a camera mounted on the camera mounting portion, the camera having a field of view, a light source mounted on the light mounting portion for illuminating at least a portion of the field of view of the camera; and at least one temperature sensor mounted on the flex circuit for measuring a temperature of the light source and a temperature of an ambient environment of the imaging assembly; and a control circuit in communication with the light source and the at least one temperature sensor, the control circuit controlling an output of the light source to control a difference between the temperature of the ambient environment and the temperature of the light source. The control circuit controls the difference between the temperature of the ambient environment and the temperature of the illumination source to a predetermined amount.

### CROSS-REFERENCE TO RELATED APPLICATION

This application claims the benefit of priority under 35 U.S.C. §119 to U.S. Patent Application No. 61/908,284, titled IMAGING CATHETER WITH THERMAL MANAGEMENT ASSEMBLY, filed on Nov. 25, 2013, the entirety of which is incorporated herein by reference for all purposes.

### BACKGROUND

Aspects of the present invention generally relate to an imaging catheter and, particularly, to an imaging feeding tube having a thermal management assembly.

Several medical procedures involve positioning a catheter, such as a feeding tube or endoscope, within a patient through the patient's nose, mouth, or other opening. In many procedures, accurately positioning the catheter is crucial to the success of the procedure and/or to the safety of the patient. For example, a nasogastric feeding tube may be inserted through the nose, past the throat, and down into the stomach, or past the stomach into the small bowels of the patient to deliver food to the patient via the tube. If the feeding tube is mistakenly positioned in the patient's lung, the feeding solution would be delivered to the patient's lung causing critical and possibly fatal results.

### SUMMARY

There is disclosed a feeding tube assembly comprising a flexible feeding tube having opposite first and second longitudinal ends, a longitudinal axis extending between the first and second longitudinal ends, and a feeding passage defined therein extending along the longitudinal axis between the first and second longitudinal ends; an imaging assembly including an imaging device and an illumination source, the imaging assembly configured for generating and transmitting imaging signals indicative of images of an alimentary canal of a subject, wherein the imaging assembly is secured to the feeding tube adjacent the first longitudinal end of the feeding tube, the illumination source being configured to illuminate an ambient environment of the imaging assembly; at least one temperature sensor configured to measure a temperature of at least the illumination source and a temperature of the ambient environment of the imaging assembly; and a control circuit in communication with the illumination source and the at least one temperature sensor, the control circuit controlling an output of the illumination source to control a difference between the temperature of the ambient environment and the temperature of the illumination source. In some cases, for example, the thermal management assembly includes at least one temperature sensor disposed to measure a temperature of a portion of the imaging catheter adjacent to heat-generating components of the catheter. The control circuit controls the difference between the temperature of the ambient environment and the temperature of the illumination source to a predetermined amount. The predetermined amount is, in some embodiments, a temperature difference of about 2 degrees Celsius. In some cases, the control circuit controls the illumination source at a maximum output of the illumination source as long as the difference between the temperature of the ambient environment and the temperature of the illumination source is maintained at a predetermined amount. In some cases, the control circuit passively controls the output of the illumination source by changing the output only after the difference between the temperature of the ambient environment and the temperature of the illumination source is detected to be greater than or less than the predetermined amount. In some cases, the control circuit actively controls the output of the illumination source by continually controlling the output of the illumination source to maintain the difference between the temperature of the ambient environment and the temperature of the illumination source at the predetermined amount. The feeding tube assembly set can further comprise a first temperature sensor for measuring the temperature of the illumination source and a second temperature sensor for measuring the temperature of the ambient environment. The first temperature sensor is typically disposed directly adjacent the illumination source, or at least one of the heat-generating components of the catheter. The second temperature sensor is typically disposed remote from the illumination source, or any of the one or more heat-generating components of the catheter. The first and second temperature sensors are typically thermistors. The feeding tube assembly can further comprise an inlet adaptor adjacent the second longitudinal end of the feeding tube in fluid communication with the feeding passage, the inlet adaptor configured for fluid connection with a source of enteral feeding liquid.

There is disclosed an imaging catheter assembly comprising an elongate body having a first body end, and an opposite second body end; an imaging assembly secured to the first body end, the imaging assembly having a first imaging assembly end remote from the first body end and a second imaging assembly end adjacent the first body end. The imaging assembly includes a flex circuit having an electronic component mounting portion, a camera mounting portion adjacent the first imaging assembly end, and a light mounting portion adjacent the first imaging assembly end; a camera mounted on the camera mounting portion, the camera having a field of view, a light source mounted on the light mounting portion for illuminating at least a portion of the field of view of the camera; and at least one temperature sensor mounted on the flex circuit for measuring a temperature of the light source and a temperature of an ambient environment of the imaging assembly; and a control circuit in communication with the light source and the at least one temperature sensor, the control circuit controlling an output of the light source to control a difference between the temperature of the ambient environment and the temperature of the light source. The control circuit controls the difference between the temperature of the ambient environment and the temperature of the illumination source to a predetermined amount. The predetermined amount is, for example, a temperature difference of about 2 degrees Celsius. The imaging catheter assembly set can further comprise a first temperature sensor for measuring the temperature of the light source and a second temperature sensor for measuring the temperature of the ambient environment. The first temperature sensor is disposed on the light mounting portion of the flex circuit adjacent the light source and the second temperature sensor is disposed on the electronic component mounting portion of the flex circuit remote from the light source. The first and second temperature sensors can be thermistors.

There is also disclosed an imaging catheter assembly comprising an elongate body having a first body end, and an opposite second body end; an imaging assembly secured to the first body end, the imaging assembly having a first imaging assembly end remote from the first body end and a second imaging assembly end adjacent the first body end, the imaging assembly including a flex circuit having an electronic component mounting portion, a camera mounting portion adjacent the first imaging assembly end, and a light mounting portion adjacent the first imaging assembly end; a camera mounted on the camera mounting portion, the camera having a field of view, a light source mounted on the light mounting portion configured to illuminate at least a portion of the field of view of the camera; and at least one temperature sensor mounted on the flex circuit for measuring at least one of a temperature of the light source and a temperature of an ambient environment of the imaging assembly; and a control circuit in communication with the light source and the at least one temperature sensor, the control circuit configured to control an output of the light source based on at least one of the temperature of the light source and the temperature of the ambient environment. The control circuit can be configured to control the output of the light source to a predetermined amount of difference between the temperature of the illumination source and the temperature of the ambient environment; the predetermined amount can be a temperature difference of about 2 degrees Celsius. The at least one temperature sensor can comprise a first temperature sensor configured to measure the temperature of the light source and a second temperature sensor configured to measure the temperature of the ambient environment. The first temperature sensor can be disposed on the light mounting portion of the flex circuit adjacent the light source. The second temperature sensor can be disposed on the electronic component mounting portion of the flex circuit remote from the light source. The first and second temperature sensors can be thermistors.

Other advantages and features will be in part apparent and in part pointed out hereinafter.

### BRIEF DESCRIPTION OF THE DRAWINGS

FIG. 1 is a schematic illustration showing a perspective view of an imaging feeding tube assembly.

FIG. 2 is schematic illustration showing a perspective view of the feeding tube assembly in FIG. 1.

FIG. 3 is a schematic illustration showing a side, elevational view of an imaging feeding tube system, including the imaging feeding tube assembly in FIG. 1, and an interface cable, and a console.

FIG. 4 is a schematic illustration showing an enlarged, fragmentary, perspective view of a distal end portion of the feeding tube assembly in FIG. 1, including an exploded imaging assembly, an imaging assembly connector, and a portion of the feeding tube.

FIG. 5 is a schematic illustration showing an enlarged cross section view of the feeding tube of the feeding tube assembly in FIG. 1.

FIG. 6 is a schematic illustration showing a top perspective view of a flex circuit assembly of the imaging assembly in FIG. 4, in a folded configuration.

FIG. 7 is a schematic illustration showing a bottom perspective view of the flex circuit assembly of the imaging assembly in FIG. 4, in the folded configuration.

FIG. 8 is a schematic illustration showing an enlarged fragmentary section view of the distal end portion of the imaging assembly in FIG. 4.

FIG. 9 is an electrical schematic of a thermal management system of the imaging assembly.

Corresponding reference characters indicate corresponding parts throughout the drawings.

### DETAILED DESCRIPTION

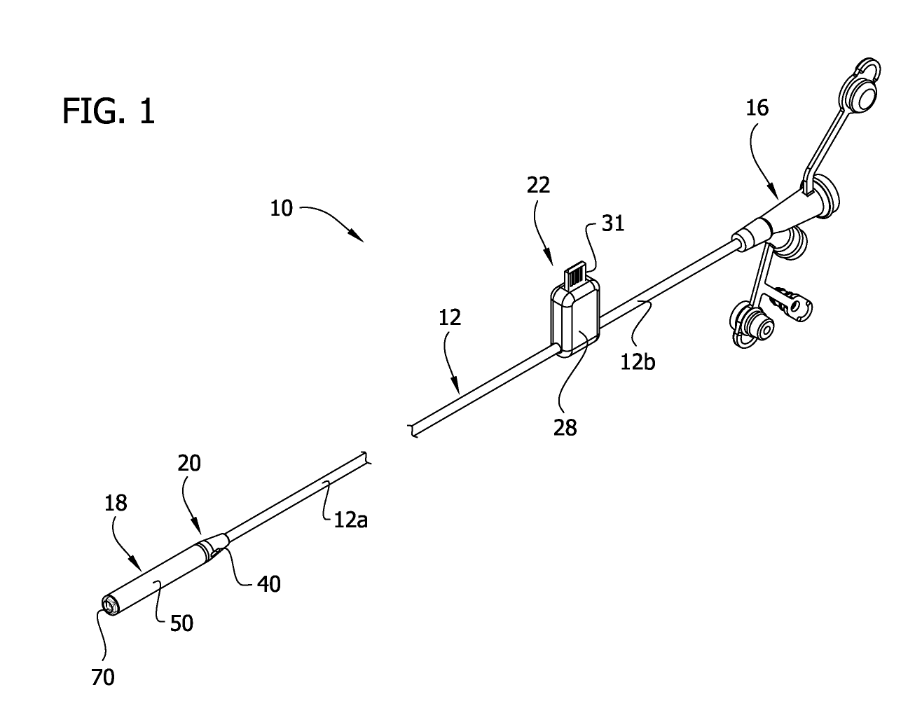

Referring now to the drawings, and in particular to FIGS. 1-3, an imaging catheter is generally indicated at 10. As disclosed herein, the imaging catheter can be a medical device that is configured for insertion into a subject (e.g., a human or a non-human subject) and configured to provide images (e.g., digital video) of anatomy of the subject as the medical device is inserted into the subject and/or after the medical device is positioned in the subject. In the illustrated embodiment, the imaging catheter is configured as a feeding tube assembly 10 and exemplarily illustrated as a nasogastric feeding tube assembly. In general, the illustrated nasogastric feeding tube assembly 10 can be configured to provide images of an alimentary canal, or a portion(s) thereof, of the subject as the feeding tube assembly is inserted into the subject and after the feeding tube assembly is positioned in the subject to facilitate confirmation of proper placement of the feeding tube assembly in the subject. The nasogastric feeding tube assembly 10 can be also configured to deliver liquid nutrients into the alimentary canal of the subject by enteral feeding, such as after a user (e.g., medical practitioner) confirms proper placement of the feeding tube assembly in the subject, by viewing the acquired digital images from the imaging feeding tube assembly. It is understood that the imaging catheter 10 may be configured as a different type of feeding tube, such as a gastric feeding tube, or a jejunostomy feeding tube, or may be configured as a different type of medical device, such as an endoscope, or a heart catheter (e.g., balloon catheter or other type of heart catheter).

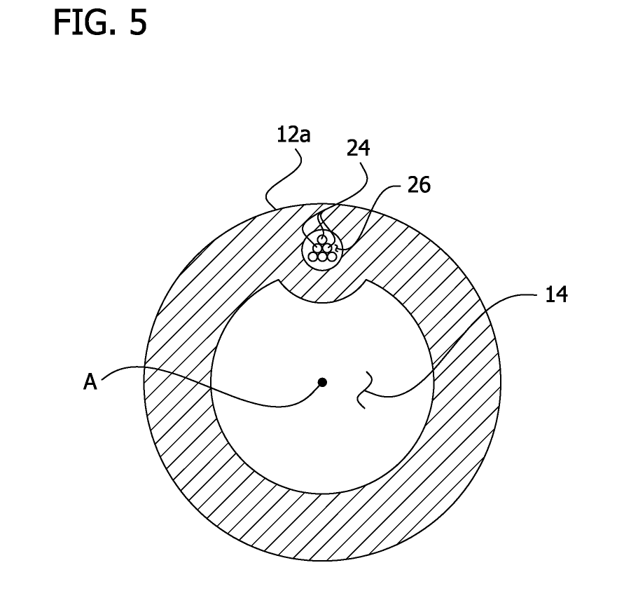

The illustrated feeding tube assembly 10 generally includes an elongate, generally flexible body in the form of a feeding tube, generally indicated at 12, having a longitudinal axis A (FIG. 5), an open first longitudinal end (i.e., a distal end) and an open second longitudinal end (i.e., a proximal end). A feeding passage 14, defined by an interior surface of the feeding tube 12, extends longitudinally between the longitudinal ends of the tube for delivering nutrients (e.g., in the form of an enteral feeding solution) to the subject. In other embodiments—such as catheters that are not feeding tubes—the elongate body may have other configurations, and may not have a longitudinal passage for delivering fluids to the patient. An inlet adapter, generally indicated at 16, for delivering liquid nutrients into the feeding passage 14 is attached to the second end of the tube, and an imaging assembly, generally indicated at 18, for generating and transmitting real time images (e.g., video) of the alimentary canal of the patient during and/or following intubation is attached to the first end of the tube 12 by an imaging assembly connector, generally indicated at 20.

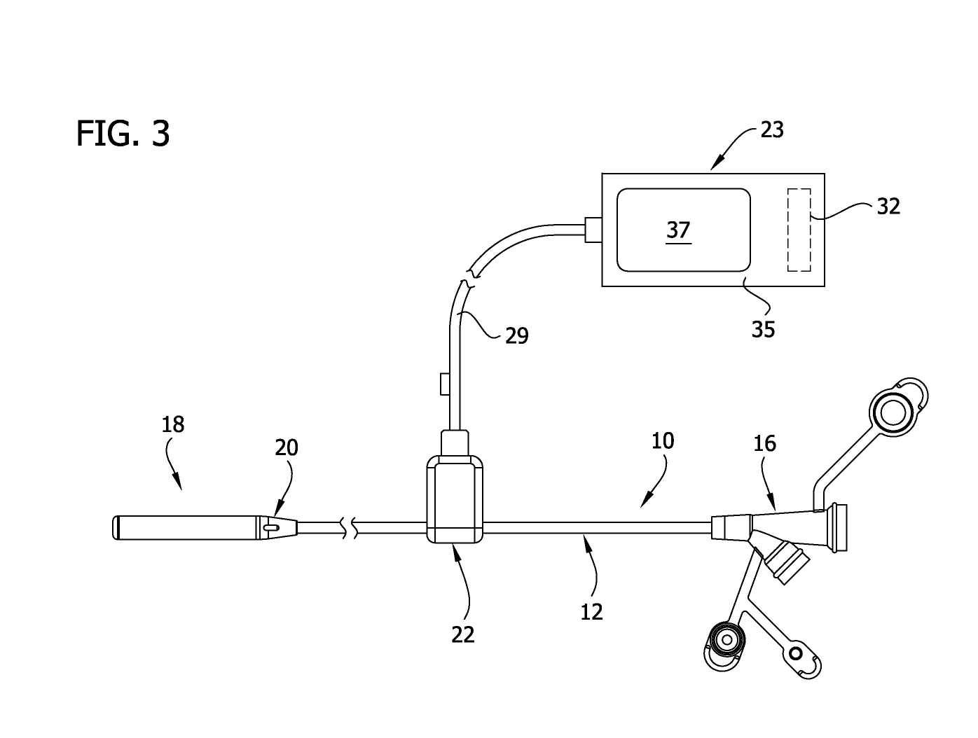

As used herein with the point of reference being the feeding source, the inlet adaptor 16 defines the proximal end of the feeding tube assembly 10, and the imaging assembly 18 defines the distal end. The feeding tube assembly 10 can also include a console connector, generally indicated at 22, in communication with the imaging assembly 18, to provide communication between the imaging assembly and a console 23 (FIG. 3), on which the images obtained by the imaging assembly 18 may be displayed. In the illustrated embodiment, the feeding tube assembly 10, the console 23, and an interface cable 29, which communicatively connects the feeding tube assembly to the console, together constitute an imaging catheter system, and more specifically, an imaging feeding tube system.

Referring to FIGS. 1-3, the exemplarily illustrated feeding tube 12 comprises two tube segments: a first tube segment 12a extending between the imaging assembly connector 20 and the console connector 22, and a second tube segment 12b extending between the console connector and the inlet adaptor 16. The first and second tube segments 12a, 12b can be secured to the console connector 22 in such a way that the first and second tube segments are in fluid communication with each other to at least partially define the feeding passage 14. In other embodiments of the disclosure, the tube 12 may be formed as an integral, one-piece component. The feeding tube 12 may be formed from a thermoplastic polyurethane polymer, such as but not limited to, an aromatic, polyether-based thermoplastic polyurethane, and a radiopaque substance, such as barium. The tube 12 may be formed from other materials without departing from the scope of the present disclosure.

As shown in FIG. 5, the first tube segment 12a of the feeding tube 12 may include one or more electrical conductors 24 typically disposed in the tube wall of the first tube segment. The electrical conductors 24 run longitudinally along the first tube segment, such as along or parallel a longitudinal axis of the feeding passage 14. At least some of the electrical conductors 24 can be configured to transmit imaging signals between the imaging assembly 18 and the console 23. Other electrical conductors 24 may be configured to transmit power from the console 23 to the imaging assembly 18, and provide a ground. Still other electrical conductors 24 may be configured to provide other communication including, but not limited to, two-way communication, between the console 23 and the imaging assembly 18. In one or more embodiments of the disclosure, at least one of the electrical conductors 24 is configured to supply power from a power supply, which can be the console 23, to the imaging assembly 18, although other ways of powering the imaging assembly, including the imaging assembly having its own source of power, do not depart from the scope of the present disclosure. As exemplarily illustrated, the electrical conductors 24 can be disposed within a conductor passage 26 of the feeding tube 12 so that the conductors are physically separated or at least fluidly isolated from the feeding passage 14 to inhibit or reduce the likelihood of feeding solution in the feeding passage from contacting the conductors.

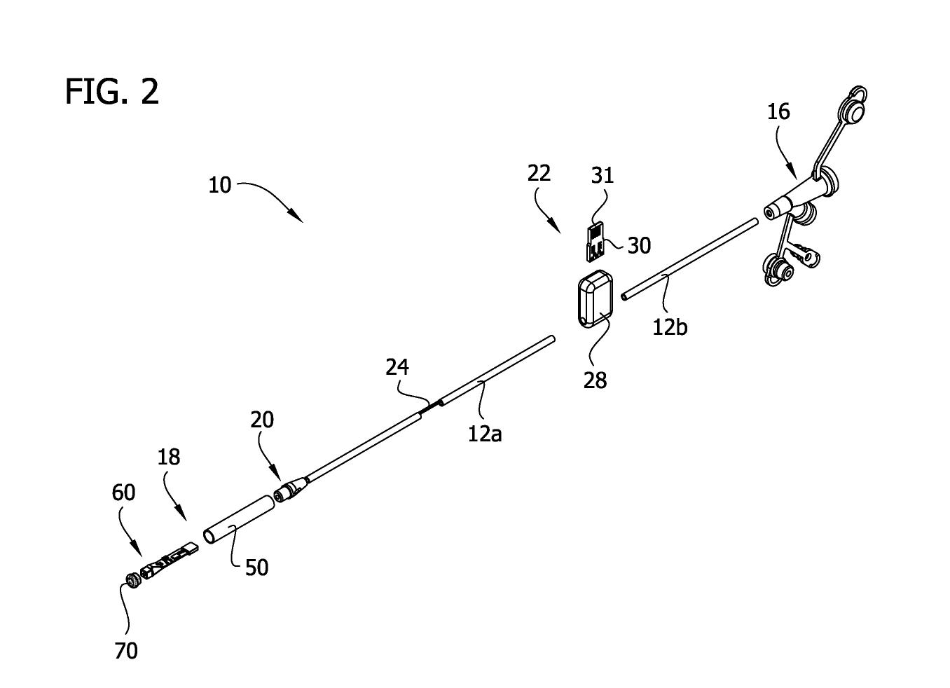

The electrical conductors 24 extend from the first tube segment 12a into a connector housing 28 of the console connector 22 and are electrically connected to a PCB 30 (FIG. 2). The interface cable 29 (or other signal-transmitting component) can be removably connectable to edge connector 31 to effect communication and data exchange between the console 23 and the imaging assembly 18. An electronic memory component, such as electrically erasable programmable read-only memory (EEPROM), may be mounted on the PCB 30 to allow information (i.e., data) to be stored and/or written thereon and to be accessible by the console (i.e., a microprocessor 32 of the console 23) or another external device. It is understood that the PCB 30 may have additional or different electrical components mounted thereon, or the PCB may be omitted such that the electrical conductors are operatively connected to the PCB 30. The console 23 can also include a console housing 35, a console display 37, such as an LCD or other electronic display, secured to the housing, and microprocessor 32 (broadly, “a control circuit”) disposed in the housing. In the illustrated embodiment, the microprocessor 32 communicates with the imaging assembly 18 through the interface cable 29 and the electrical conductors 24.

Referring to FIGS. 1, 2, and 4 the imaging assembly connector 20 may define a feeding outlet 40 that is in fluid communication with the feeding passage 14 of the tube 12. The feeding outlet 40 can comprise one or more openings extending laterally through a side of the imaging assembly connector 20 (only one such lateral opening is illustrated). In the illustrated embodiment, the first or distal end of the tube 12 is received and secured within the imaging assembly connector 20 at a proximal end of the imaging assembly connector to provide fluid communication between the feeding passage 14 and the feeding outlet 40. When the feeding tube assembly 10 is determined to be appropriately positioned in a patient, feeding solution or other desirable liquid fed into the inlet adaptor 16 can be introduced through the feeding passage 14 of the tube 12, and out through the outlet 40 and into the subject's alimentary canal.

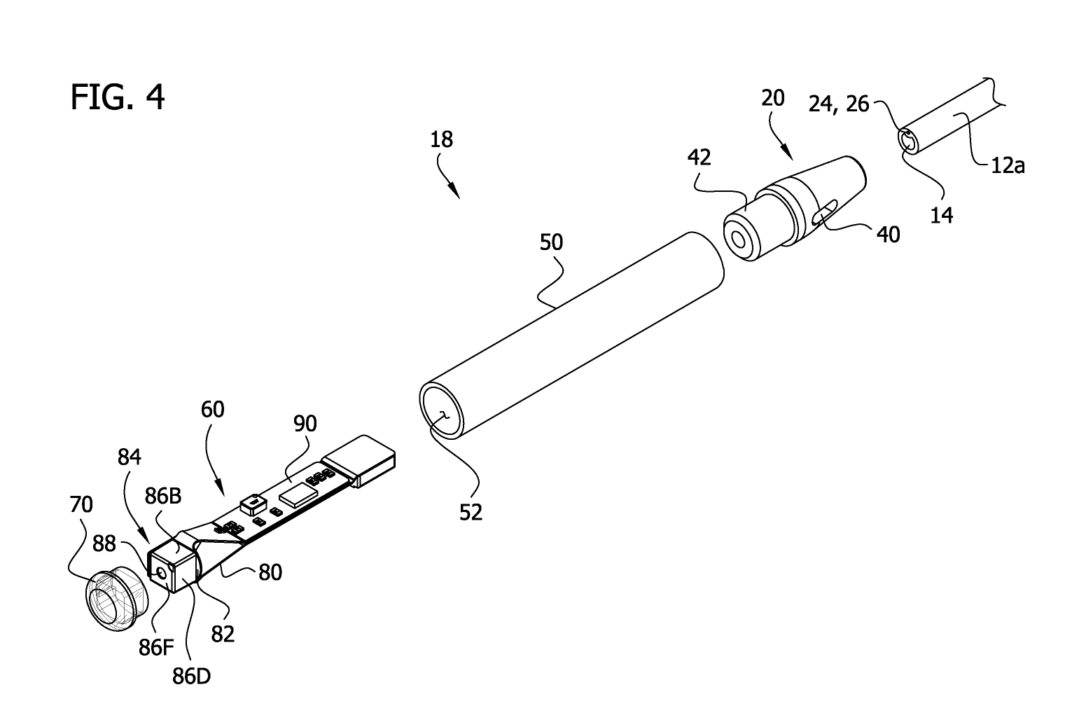

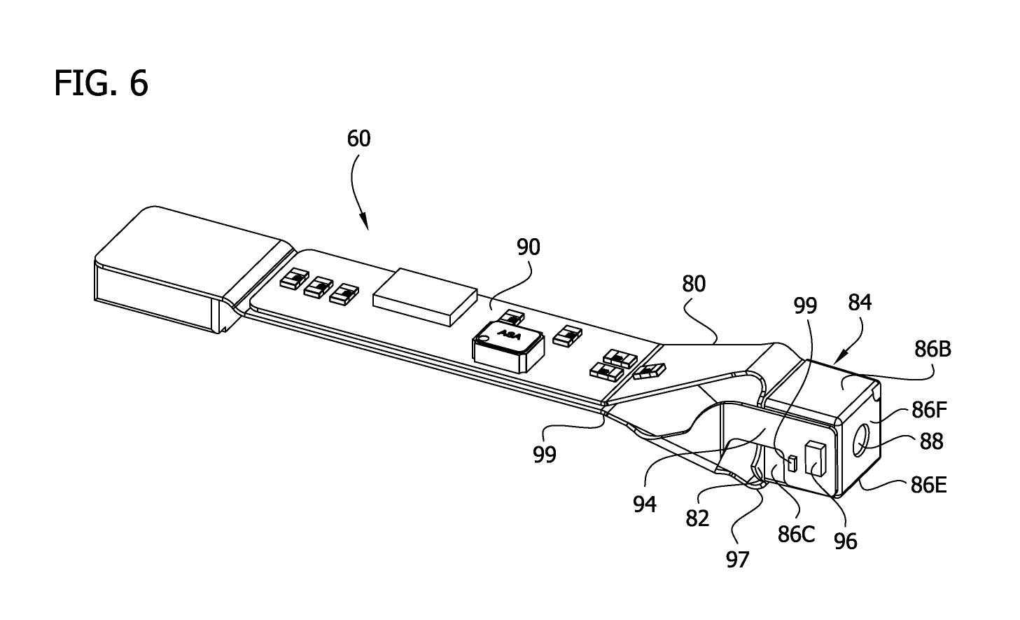

Referring to FIG. 4, the imaging assembly 18 can include a tubular housing 50, a flexible circuit (“flex circuit”) assembly 60 disposed within the tubular housing, and a transparent or translucent cap 70 secured to the tubular housing 50. Generally speaking a flex circuit includes a deformable circuit element and components mounted on the deformable circuit element. The deformable circuit element may be a flat (at least prior to being deformed) substrate that can be bent or otherwise deformed, and which also includes electrical conductors for making electrical connection among various components that may be mounted on the substrate. The deformable circuit element may only be partially deformable (e.g., only at discrete bend lines) within the scope of the present disclosure. Among other functions, the tubular housing 50 can provide protection for the flex circuit assembly 60, and the housing may be substantially waterproof to inhibit the ingress of liquid into the imaging assembly 18. The tubular housing 50 has an interior surface defining an axial passage 52 shaped and sized for housing the flex circuit assembly 60 in a folded configuration. In one embodiment, the tubular housing 50 is formed from a generally flexible material that provides protection for the flex circuit assembly 60 and allows the imaging assembly 18 to bend to facilitate maneuverability of the feeding tube assembly 10. A second end, such as a proximal end, of the tubular housing 50 can be configured to receive a connection portion 42 of the imaging assembly connector 20, and can be adhered thereto to secure the imaging assembly to feeding tube 12. The tubular housing 50 may be generally opaque, by being formed from an opaque white material or having an opaque material applied thereon, to reflect illumination from a light source, such as an internal LED 96 (FIG. 6), and direct the illumination outward from the distal end of the imaging assembly 18 to, for example, a field of view.

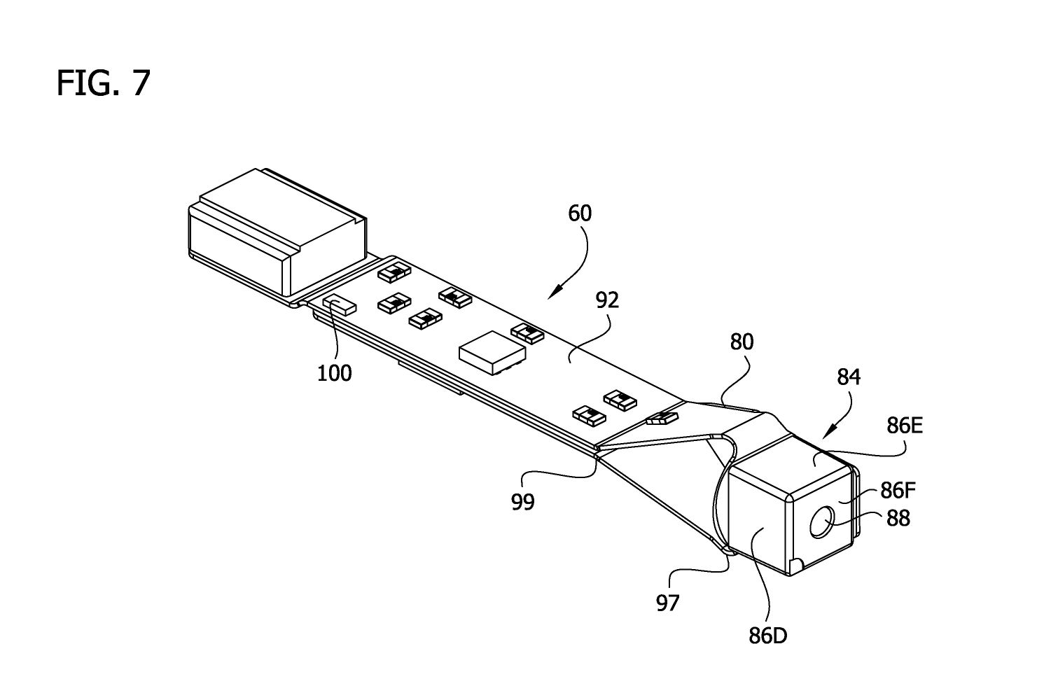

The flex circuit assembly 60 typically includes a flex circuit 80 and electronic components (not labeled), described below, attached thereto. In the partially assembled or folded configuration exemplarily shown in FIGS. 4, 6, and 7, the flex circuit assembly 60 can have a length with a first longitudinal end, e.g., a distal end, and an opposite second longitudinal end, e.g., a proximal end. The electrical conductors 24 can be connected to the second longitudinal end, e.g., the proximal end, of the flex circuit assembly 60. A camera mounting portion 82 is typically disposed at the first longitudinal end, e.g., the distal end of the flex circuit assembly 60. An imaging device such as a digital camera, generally indicated at 84, can be mounted on the camera mounting portion 82. The camera 84 can have a cuboidal housing 86 with a base 86A, as shown in FIG. 7, sides 86B, 86C, 86D, 86E, and an upper or first surface 86F. The distal end surface 86F of the camera 84 can include a lens 88. The lens 88 defines a field of view that projects generally outward from the distal end of the imaging assembly 18. In accordance with one or more embodiments of the disclosure, the camera 84 comprises an imaging device, such as a CMOS imaging device. In further embodiments of the disclosure, the camera 84 may comprise a different type of solid state imaging device, such as a charge-coupled device (CCD), or another type of imaging device. Other ways of configuring the electronics and other components of the imaging assembly 18 do not depart from the scope of the present disclosure and may be implemented as variant embodiments thereof. For example, in another embodiment, the flex circuit assembly 60 may be replaced with a rigid printed circuit board (PCB). Moreover, it will be understood that an optical imaging assembly (not shown) may be used.

The flex circuit assembly 60 can include a power mounting portion 90 (FIGS. 4 and 6) and a control or data mounting portion 92 (FIG. 7) each typically extending from the camera mounting portion 82 at a fold line toward the first longitudinal end of the flex circuit assembly 60. Power supply components are typically disposed on the power mounting portion 90, and camera control components are typically disposed on the data mounting portion 92.

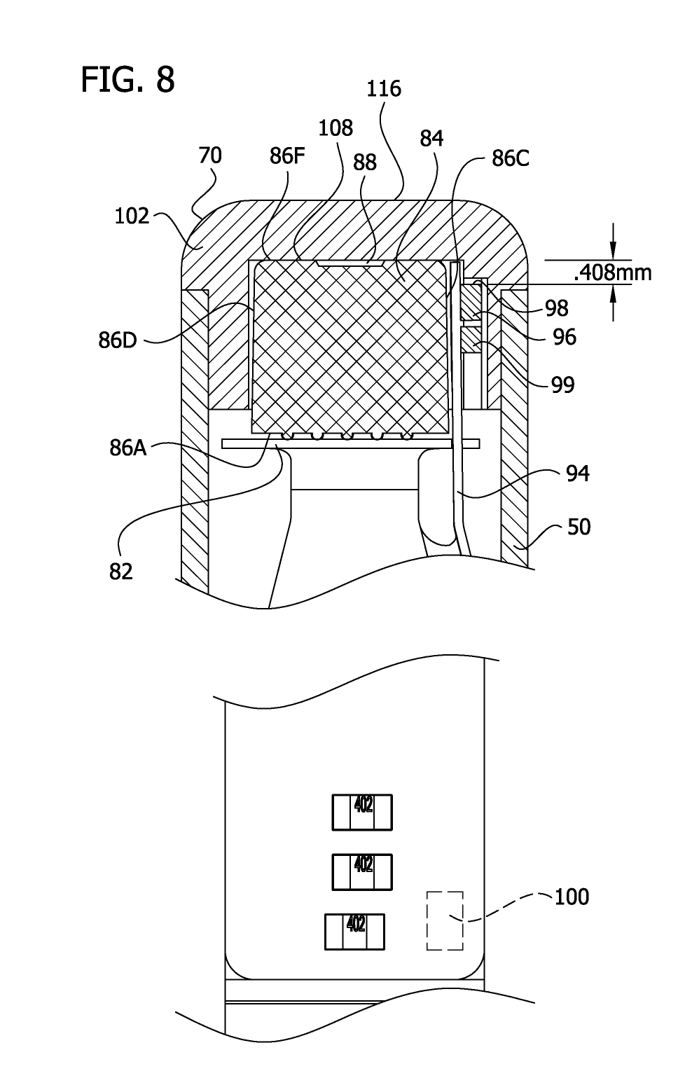

Referring to FIGS. 6 and 8, a light mounting portion 94 of the flex circuit 60 can be disposed at the side 86C of the camera 84. The light mounting portion 94 is illustratively depicted as extending longitudinally toward the camera 84 from a lateral side edge of the flex circuit at a fold line of the power mounting portion 90. One or more light sources 96 can be disposed on, for example, the light mounting portion 94 for illuminating an area or region adjacent to the distal end surface 86F of the camera housing 86. In the illustrated embodiment, the light source is a light emitting diode (LED) 96 disposed on the light mounting portion 94 so that the LED is disposed on the side 86C of the camera housing and below or proximate the upper surface 86F of the camera housing. In the illustrated embodiment, the LED 96 has a light emitting surface 98 substantially perpendicular to the light mounting portion 94 for projecting light outward from the distal end of the imaging assembly 18. According to the illustrated embodiment (FIG. 8), the LED 96 and the light mounting portion 94 are positioned relative to the camera 84 and the camera mounting portion 82 such that the light emitting surface 98 of the LED 96 is a relatively short distance (e.g., 0.408 millimeters) below the upper surface 86F of the camera housing 86. Typically, LED 96 has an illumination zone that is at least partially coincident over an imaging zone or field of view of camera 84, through optional lens 88.

A light source temperature sensor 99 may be disposed on the light mounting portion 94 adjacent the LED 96. The light source temperature sensor 99 is configured to measure a temperature of the LED 96. An ambient temperature sensor 100 may be disposed on the control mounting portion 92. However, it is envisioned that the ambient temperature sensor 100 could be disposed at other locations on or adjacent the flex circuit assembly 60. The ambient temperature sensor 100 is configured to measure a temperature of the ambient environment around the imaging assembly 18. As will be explained in greater detail below, measuring both the light source temperature and the ambient temperature allows for a determination of the difference between the two temperatures for regulating the difference between the two temperatures during use of the imaging catheter 10.

Operation of the LED 96 to illuminate the field of view of lens 88 may cause the temperature of the LED to exceed that of the ambient environment around the imaging assembly 18 by more than a desirable amount. To ensure the difference between the ambient temperature and the light source temperature does not fluctuate away from an acceptable amount while maintaining the maximum output of light for viewing, controller 32 may selectively control an output of the LED 96. In particular, the controller 32 may control the output of the LED 96 by controlling the power supplied by a power source (e.g., console 23) to the LED. A PWM driver may also be used to drive the LED 96 and the controller 32 may control the PWM driver to control the output of the LED.

As mentioned above, controlling the output of the LED 96 may be used to control the difference between the ambient temperature and light source temperature detected by the ambient temperature sensor 100 and light source temperature sensor 99, respectively. For example, if the temperature sensors 99, 100 detect respective temperatures having a difference other than a predetermined amount, the controller 32 can adjust (i.e., increase or decrease) the output of the LED 96 to regulate the temperature difference between the ambient environment around the imaging assembly and the LED 96. Alternatively, the controller 32 may continually control the power supplied to the LED 96 to continually control the output of the LED so that the difference between the ambient temperature and the light source temperature remains at a predetermined amount. In this instance, power can be increased and decreased as needed to keep the difference between the ambient temperature and the light source temperature at the predetermined amount. The controller 32 can include a control loop mechanism such as a PID controller to maintain the difference between the ambient environment and the LED 96 at the predetermined amount. In it envisioned that both analog and digital control loops can be used within the scope of the present disclosure.

In a preferred embodiment the controller 32 maintains the temperature difference between the ambient environment and the LED 96 to about 2 degrees Celsius. The controller may alternatively maintain the temperature difference within a predetermined range. The range may be centered on a temperature difference of about 2 degrees Celsius. The controller 23 may maintain the temperature difference at other values within the scope of the present disclosure. In some cases, the temperature difference is about 1 degree Celsius.

In a situation where the imaging catheter is operating in a relatively cold environment or an environment where there is relatively little heat transfer from the LED 96, the difference between the temperature of the LED and the ambient temperature around the imaging assembly 18 may increase above the predetermined amount. In this instance the temperature difference is a positive difference where the temperature of the LED 96 is greater than the ambient temperature. Should the temperature difference exceed the predetermined amount, the controller 32 may decrease the output of the LED 96 to decrease the temperature of the LED to restore the desired temperature difference between the LED and the ambient environment.

If the imaging assembly 18 enters an area of the body that provides a substantial heat sink for the LED 96, the LED temperature detected by the LED temperature sensor may fall below the predetermined temperature differential and possibly even below the ambient temperature detected by the ambient temperature sensor 100. The controller 32 may increase the output of the LED 96 up to a maximum output to permit the most light possible for viewing, while monitoring any resultant temperature change in the LED temperature sensor 99. It will be understood that the output of the LED 96 can be increased while still maintaining the temperature difference at the predetermined amount. This allows the imaging catheter 10 to operate with a maximum light permissible output from the LED 96 at all times.

The amount in which the temperature difference exceeds the desired amount may also control the rate and/or extent to which the output of the LED 96 is increased or decreased. Thus, a large temperature difference above or below the desired amount may result in a significant increase or decrease in the output of the LED 96. In the instance where a significant decrease in the output of the LED 96 is required, the controller 23 may completely shut off the output of the LED (i.e., turn off all power to LED). Conversely, when a significant increase in the output of the LED 96 is required, the controller 23 may supply maximum power to the LED.

In some embodiments, the controller 32 may reduce the power supplied to the LED to reduce the output of the LED in order to reduce the ambient temperature around the imaging assembly 18 if the ambient temperature sensor 100 detects an ambient temperature above a predetermined threshold. Alternatively, the controller 32 may continually control the power supplied to the LED 96 to continually control output of the LED so that the ambient temperature remains below the predetermined threshold. In this instance, power can be increased and decreased as needed to keep the ambient temperature below the predetermined threshold. In a preferred embodiment, the controller 32 controls the output of the LED 96 to a maximum output of the LED (i.e., maximum power supplied to the LED) as long as the ambient temperature measured by the ambient temperature sensor 100 remains below the predetermined threshold.

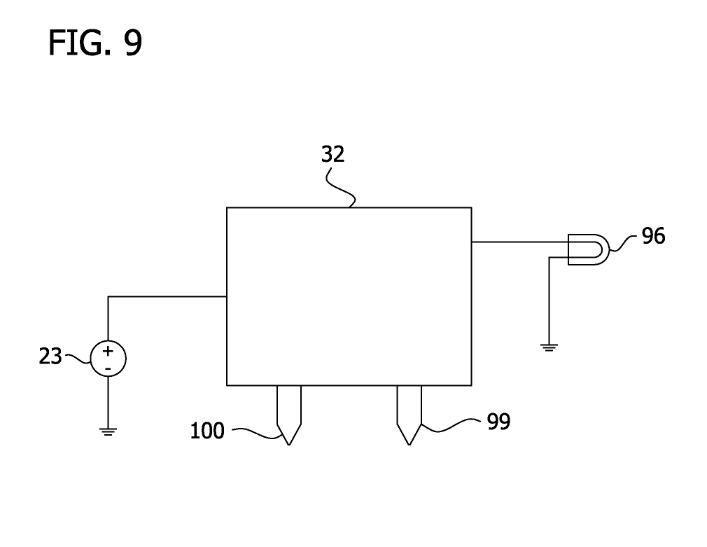

In the illustrated embodiment, the temperature sensors 99, 100 are thermistors. However, other types of temperature sensors are envisioned. Further, it is envisioned that a single temperature sensor can be used to measure both the ambient temperature and the temperature of the LED 96. The console (i.e., power supply) 23, controller 32, LED 96, light source temperature sensor 99, and ambient temperature 100 may be broadly considered a thermal management system (FIG. 9).

In other cases, the control circuit can be configured to regulate the output, e.g., the power, of the light source, e.g., any one or all of the LEDs, based on the temperature of the light source, or portion thereof, or based on the ambient temperature. For example, the controller can be configured to regulate the output of the light source to a predetermined temperature of the light source that is less than about 40 degrees Celsius, e.g., the predetermined temperature can be in a range of from about 37 degrees Celsius to about 40 degrees Celsius.

As various changes could be made in the above constructions and methods without departing from the scope of the invention, it is intended that all matter contained in the above description and shown in the accompanying drawings shall be interpreted as illustrative and not in a limiting sense. For example, one or more aspects of the invention can involve regulating operation of any heat-generating component of the imaging catheter, including the one of more LEDs 96 and any of the components on any of the power mounting portion 90 and the control or data mounting portion 92. Thus, thermal management can involve regulation of the operation of any heat-generating component of an imaging catheter assembly or system. Further, a representation of the ambient temperature can be utilized as a proxy or an approximation of the actual ambient temperature. Accordingly, when used herein the term “ambient temperature” is intended to include such a representation. Further, the ambient temperature, including the representation of the ambient temperature, can involve a surface temperature of any outside or wetted surface of the assembly that is intended or expected to be in contact with a subject, e.g., the subject's alimentary canal. Thus, in some cases, the controller can be configured to regulate the output of the light source to a predetermined ambient temperature that is less than about 40 degrees Celsius, e.g., the predetermined outside surface temperature of the assembly can be in a range of from about 37 degrees Celsius to about 40 degrees Celsius.

In still further configurations, the controller is further configured to provide an indication that any of the temperature of the light source and the ambient temperature is at a predetermined temperature. For example, the controller can be configured to energize an indicator, e.g., a warning light, or to provide a signal to the console 23 which can show on the console display 37 thereof any of the warning indication, the measured light source temperature, and the measured ambient temperature.

When introducing elements of the present invention or the preferred embodiments(s) thereof, the articles “a”, “an”, “the” and “said” are intended to mean that there are one or more of the elements. The terms “comprising”, “including” and “having” are intended to be inclusive and mean that there may be additional elements other than the listed elements.

In view of the above, it will be seen that the several objects of the invention are achieved and other advantageous results attained.

1-15. (canceled)

16. A feeding tube assembly comprising:
a flexible feeding tube having opposite first and second longitudinal ends, a longitudinal axis extending between the first and second longitudinal ends, and a feeding passage defined therein extending along the longitudinal axis between the first and second longitudinal ends;
an imaging assembly including an imaging device and an illumination source, the imaging assembly configured for generating and transmitting imaging signals indicative of images of an alimentary canal of a subject, wherein the imaging assembly is secured to the feeding tube adjacent the first longitudinal end of the feeding tube;
at least one temperature sensor configured to measure a temperature of at least a portion of the imaging assembly and a temperature of an ambient environment of the imaging assembly; and
a control circuit in communication with the illumination source and the at least one temperature sensor, the control circuit configured to control an output of the illumination source based on a difference between the temperature of the ambient environment and the temperature of the at least a portion of the imaging assembly.

17. The feeding tube assembly set forth in claim 16, further comprising an inlet adaptor disposed adjacent the second longitudinal end of the feeding tube in fluid communication with the feeding passage, the inlet adaptor configured for fluid connection with a source of enteral feeding liquid.

18. The feeding tube assembly set forth in claim 16, wherein the control circuit is configured to control the illumination source at a maximum output of the illumination source as long as the difference between the temperature of the ambient environment and the temperature of the at least a portion of the imaging assembly is maintained at a predetermined amount.

19. The feeding tube assembly set forth in claim 16, wherein the control circuit is configured to control the difference between the temperature of the ambient environment and the temperature of the at least a portion of the imaging assembly to be less than or equal to a predetermined amount.

20. The feeding tube assembly set forth in claim 17, wherein the temperature of the at least a portion of the imaging assembly is a temperature of the illumination source.

21. The feeding tube assembly set forth in claim 20, wherein the control circuit is configured to control the output of the illumination source by changing the output only after the difference between the temperature of the ambient environment and the temperature of at least a portion of the imaging assembly is detected to be greater than or less than the predetermined amount.

22. The feeding tube assembly set forth in claim 20, wherein the control circuit is configured to control the output of the illumination source by continually controlling the output of the illumination source to maintain the difference between the temperature of the ambient environment and the temperature of the at least a portion of the imaging assembly to be less than or equal to the predetermined amount.

23. The feeding tube assembly set forth in claim 22, wherein the predetermined amount is a temperature difference of about 2 degrees Celsius.

24. The feeding tube assembly set forth in claim 16, wherein the at least one temperature sensor comprises a first temperature sensor configured to measure the temperature of the illumination source and a second temperature sensor configured to measure the temperature of the ambient environment.

25. The feeding tube assembly set forth in claim 24, wherein the first temperature sensor is disposed directly adjacent the illumination source.

26. The feeding tube assembly set forth in claim 25, wherein the second temperature sensor is disposed remotely from the illumination source.

27. The feeding tube assembly set forth in claim 26, further comprising an inlet adaptor disposed adjacent the second longitudinal end of the feeding tube in fluid communication with the feeding passage, the inlet adaptor configured for fluid connection with a source of enteral feeding liquid.

28. An imaging catheter assembly comprising:
an elongate body having a first body end, and an opposite second body end;
an imaging assembly secured to the first body end, the imaging assembly having a first imaging assembly end remote from the first body end and a second imaging assembly end adjacent the first body end, the imaging assembly including:
a flex circuit having an electronic component mounting portion, a camera mounting portion adjacent the first imaging assembly end, and a light mounting portion adjacent the first imaging assembly end;
a camera mounted on the camera mounting portion, the camera having a field of view,
a light source mounted on the light mounting portion configured to illuminate at least a portion of the field of view of the camera; and
at least one temperature sensor mounted on the flex circuit for measuring at least one of a temperature of the light source and a temperature of an ambient environment of the imaging assembly; and
a control circuit in communication with the light source and the at least one temperature sensor, the control circuit configured to control an output of the light source based on at least one of the temperature of the light source and the temperature of the ambient environment.

29. The imaging catheter assembly set forth in claim 28, wherein the control circuit is configured to control the output of the light source to a predetermined amount of difference between the temperature of the illumination source and the temperature of the ambient environment.

30. The imaging catheter assembly set forth in claim 29, wherein the at least one temperature sensor comprises a first temperature sensor configured to measure the temperature of the light source and a second temperature sensor configured to measure the temperature of the ambient environment.

31. The imaging catheter assembly set forth in claim 30, wherein the first temperature sensor is disposed on the light mounting portion of the flex circuit adjacent the light source.

32. The imaging catheter assembly set forth in claim 31, wherein the second temperature sensor is disposed on the electronic component mounting portion of the flex circuit remote from the light source.
